(19) United States
(12) Patent Application Publication
(10) Pub. No.: US 2009/0275875 A1
(43) Pub. Date: Nov. 5, 2009

(54) EXTRACORPOREAL CIRCULATION SYSTEM, ESPECIALLY FOR A MULTIFUNCTIONAL HEART-LUNG BYPASS AND FOR THE MINIMIZATION OF AIR EMBOLISMS

(75) Inventors: Kai Liebing, Jena (DE); Thorsten Wahlers, Koln (DE); Mirko Kaluza, Jena (DE)

Correspondence Address: MICHAEL J. STRIKER, 103 EAST NECK ROAD, HUNTINGTON, NY, 11743, US

(73) Assignee: MAQUET CARDIOPULMONARY AG, HIRRLINGEN (DE)

(21) Appl. No.: 11/915,298

(22) PCT Filed: Jun. 16, 2006
(86) PCT No.: PCT/EP2006/005816

§ 371 Date: Nov. 21, 2007

(30) Foreign Application Priority Data
Jun. 21, 2005 (DE) 10 2005 029 682.3

(51) Int. Cl. A61M 1/16; A61M 1/32

(52) U.S. Cl. 604/6.09; 604/6.15; 604/6.11

(57) ABSTRACT

The task was to create a multifunctional system for the extracorporeal circulation (ECC) for a heart-lung bypass, which primarily makes use of the advantages of a minimized ECC system, but which in the case of need at any time can also be used universally and at low risk as another closed and open ECC system. Besides, it shall provide the option of an automatic negative pressure and volume compensation when being used as minimized ECC system.

According to the invention, a special reservoir (48), particularly a venous cardiotomy reservoir, for blood and/or priming solution is presented, which consists of a form-stable bottom part (57), wherein a level sensor (57) can be integrated, and a flexible part (56) located on top of it, which seeks to increase its volumetric capacity.

The equipment is used in the artificial circulation of patients by the use of a heart-lung machine.

The invention relates to an extracorporeal circulation system (ECC system) with a reservoir, in particular a venous cardiotomy reservoir for blood and/or priming solution, as well as preferably with means for defoaming and filtering of the blood as it is used above all in a heart-lung bypass.

Such an ECC system is used, among others, in order to take blood, for instance, from the right atrium of the heart and feed it back into the arterial circulation of the patient via the ECC system. In this way, the function of the heart and/or the lung shall be replaced artificially, either in part or completely.

The term “Extracorporeal Circulation (ECC)” covers all methods in which the blood flows in a controlled way through an artificial circulation system outside the body, wherein its properties are selectively influenced (D. Buchwald: Extrakorporale Membranoxygeneration bei erwachsenen Patienten, 4. Technische Durchführung der extrakorporalen Oxygenation, Extrakorporale Zirkulation in Theorie und Praxis, published by Rudolf J. Tschaut, Pabst Science Publishers, 2005, p. 434). During the ECC, the heart-lung machine (HLM) technically simulates especially the pumping function of the heart and the gas exchange of the lung without taking into account the specific structure of the organs. The basic system of a heart-lung machine as used everywhere today consists of an oxygenerator (predominantly a membrane oxygenerator), which subjects the venous blood coming from the patient to a gas exchange, and a blood pump, which pumps the blood through the oxygenerator via a perfusion cannula back into the vascular system (mainly into the ascending aorta) of the patient (H. H. Weitkemper, D. Troitzsch, W. Böttcher, R. Körfer: Elemente und Funktionsprinzip einer Herz-Lungen-Maschine, Extrakorporale Zirkulation in Theorie und Praxis, published by Rudolf J. Tschaut, Pabst Science Publishers, 2005, p. 258). These perfusion systems are often generally classified in open ECC systems, closed ECC systems and minimized ECC systems, with every system having its special applications. The advantage of the open ECC system is above all that even large air bubbles in the venous tube line, particularly through the open hard-shell reservoir, are for the most part automatically eliminated. This type of system is suitable, for instance, for surgery of the cardiac valves, where the cavities of the heart are opened, which involves a particularly high risk that air enters the heart-lung machine through the venous catheters. Disadvantageous, however, are the permanent blood/air contact in the venous hard-shell reservoir even if no air enters the venous line, and the high dilution of the blood by the priming solution in the reservoir. On the other hand, it is necessary to have a high level of blood and/or priming solution in the hard-shell reservoir to prevent the blood pump from aspirating air particularly at a high pump flow. The blood collected by the reservoir and diluted with the priming solution is then conveyed by the pump into the so-called artificial lung of the heart-lung machine, the oxygenerator, and afterwards further into the arterial vascular system of the patient. In this process, only so much blood is pumped as is drained passively by gravity. This, however, is sometimes insufficient for a good circulation of the patient. On perfusion systems with active drainage, the reflux of venous blood can be improved; to this end, a negative pressure of −40 mmHg to maximally −75 mmHg is fully sufficient, and greater negative differences are to be avoided.

On the closed ECC system, in place of a venous rigid hard-shell reservoir, a soft-bag reservoir is used, which easily collapses when the blood pump aspirates a higher volume than is passively drained by gravity from the venous vascular system of the patient (H. Frerichs: Schlauchsysteme, Extrakorporale Zirkulation in Theorie und Praxis, published by Rudolf J. Tschaut, Pabst Science Publishers, 2005, p. 289). In particular, it is an advantage that there is no or almost no blood/air contact in the closed ECC system. However, it is a disadvantage that air bubbles in the venous line are not automatically eliminated in the closed reservoir. With large and many bubbles in the venous line, there is the risk that the soft bag fills with so much air that air bubbles are aspirated by the blood pump. Like with the open ECC systems, also on these closed ECC systems blood is highly diluted with the priming solution in the reservoir, which may result in the need for a foreign blood transfusion.

To minimize the dilution of the blood with priming solution, avoid the transfusion of foreign blood, and reduce the blood contact with foreign surfaces and the heparin dose, minimized ECC systems were developed. On these systems, mostly one does without a venous reservoir or uses a hard-shell or soft-bag reservoir arranged, manually completely clamped out, in the bypass of the venous line and used for filling the minimized ECC system with priming solution or for manual volume administration. Often, only the venous system of the patient is used as venous reservoir, this requiring a basically more expensive and adapted anesthesia, volume and perfusion management compared to the ECC systems described before. Here, venous blood is not drained passively by gravity, but actively through the arterial blood pump. This involves considerable risks (air embolisms) that must be taken into account. The venous cannulation place should be sealed absolutely air-tightly by a mersilene tape around the right atrium. But other sources of air embolism (for instance, central venous catheters), too, must be sealed to the atmosphere during the entire perfusion process. As a result of the aspiration of air, it may happen that the blood pump no longer delivers any volume or that it pumps microfoam into the oxygenerator and micro air bubbles into the arterial vascular system of the patient. The elimination of this air requires an intermittent pump stop (M. Kaluza, K. Liebing, T. Wahlers: Minimierte EKZ-Systeme, Extrakorporale Zirkulation in Theorie und Praxis, published by Rudolf J. Tschaut, Pabst Science Publishers, 2005, p. 296-304).

On a minimized ECC system newly introduced in the market in June 2005 (ROCSAFE, Terumo® brochure), for instance, the speed of the blood pump is automatically reduced and the venous line clamped out, when air bubbles are detected, which results in a circulatory standstill. Then, the macro bubbles are evacuated. Subsequently, the clamp is opened again manually and the speed of the centrifugal pump increased. If the next air bubble of more than 0.5 ml volume is detected, this process is repeated. The electronic clamp again closes the venous line, which again will lead to a circulatory standstill of several seconds. With activated automatic clamp, regular air aspiration may mean for the patient that continuous circulatory standstills are occurring. In order to prevent that, for instance, post-operatively any neurological problems occur due to underperfusion, the cause for the air aspiration should be found and eliminated as fast as possible. On this new minimized ECC system, a facility for automatic volume compensation does not exist, and transforming it into an open ECC system, requires a laborious mechanical and cost-intensive conversion of the ECC. Furthermore, there is the risk that when the venous catheter is aspirated (due to the general inertia of the centrifugal blood pumps and the control algorithms) excessive negative pressures are generated for a short time.

Minimized ECC systems are used, for instance, within cardiopulmonary reanimation on intensive care units (e.g. as ECMO in the case of pulmonary failure) or in aortocoronary bypass surgery, if the cavities of the heart are not opened and the risk of the entry of air in the venous line appears to be low. A particular disadvantage of these minimized ECC systems is that they usually do not provide sufficient negative pressure limitation in the venous line and no automatic volume compensation. Centrifugal and diagonal pumps can produce a negative pressure of up to −600 mmHg (e.g. DeltaStream®, Medos AG) depending on their hydraulic capacity. Based on practical experience and first own investigations in the animal experimental center, it can be assumed that heavy negative pressure fluctuations in perfusion systems with active venous drainage through the arterial blood pump may be transmitted directly to the heart and possibly even in attenuated form up to the left ventricle, for instance, through a persisting foramen ovale. The consequence may be cardial air aspiration, particularly in bypass surgery, through the opened and not severely stenosed artery or through the combined cardioplegia/vent cannula with consecutive air embolisms, organ ischemias and cerebral infarcts after the removal of the aortic clamp (K. Liebing, M. Kaluza, J. Wippermann, U. Stock, T. Wahlers: “Linksventrikuläre Luftaspirationsgefahr bei Perfusionssystemen mit direkter venöser Drainage durch die arterielle Blutpumpe”. Kardiotechnik 1/2004, p. 9-10). Though air embolisms might possibly be minimized also through such measures as carbon dioxide insufflation, short occlusion of the coronary artery (e.g. Tourniquet occlusion with Prolene 3-0) or the administration of blood cardioplegia at room temperature instead of at 34° C., it seems to be most useful, however, to achieve it by negative pressure regulation. Certainly, there is the risk that also with ECC systems with passive venous drainage, e.g. through the use of vent suction, air enters the aortic root and the left ventricle, but presumably it is much lower than with minimized ECC systems without sufficient negative pressure limitation. The adverse effect of these strong negative pressure differences to the organism is surely much greater than assumed so far. The stronger the right atrial negative pressure variations, the higher is probably the risk of left ventricular air aspiration. Except for the publications of our own cardiac surgery/cardiac engineering team, no papers were published by experts so far. Therefore, further investigations are intended. Minimized ECC systems with automatic negative pressure and volume compensation have already been suggested and partly be tested on a model (DE 103 53 418.0 as well as lecture by K. Liebing: “Air embolism in mini bypass systems”, June 2004; Stöckert Workshop; Verona/Italy).

The experiences gained so far in the application of minimized ECC systems suggest that indeed positive results have been obtained, but that this technique is just beginning to become established for daily routine use and the optimum system has not yet been found (M. Kaluza, K. Liebing, T. Wahlers: Minimierte EKZ-Systeme, Extrakorporale Zirkulation in Theorie und Praxis, published by Rudolf J. Tschaut, Pabst Science Publishers, 2005, p. 302). Development potential exists above all in the field of negative pressure regulation, the introduction of intelligent venous air bubble traps, the processing of suction blood directly on the heart-lung machine and the development of adaptive, expandable systems. This may become necessary, if the surgical situation requires an extension of the operation (e.g. replacement of ascending aorta or the aortic arch because of intraoperative dissections, laceration of the atrium, aortic laceration) and thus an upgrading of the perfusion equipment used. Here systems are in demand then, that allow such an extension without a stop of the perfusion circuit and without the corresponding risks to the patient (M. Kaluza, K. Liebing, T. Wahlers: Minimierte EKZ-Systeme, Extrakorporale Zirkulation in Theorie und Praxis, published by Rudolf J. Tschaut, Pabst Science Publishers, 2005, p. 303)

In summary, it can be stated that several ECC systems of different types are known to specialists, which satisfy specific application criteria and are specifically used according to the respective conditions existent.

However, it is not seldom that it becomes necessary (for instance, in the case of unforeseeable events and complications) to change or convert the intended or used ECC system within a very short time despite the risks to the patient (for instance, circulatory standstill, infections by unsterility, air embolisms) that are undoubtedly involved with it and the high material costs.

Therefore, the invention aims at creating a universally applicable extracorporeal circulation system, particularly for an artificial heart-lung bypass, which makes use of the advantages (such as minimum dilution and minimum foreign surface contact of blood) of a minimized ECC system, but which, if necessary and as fast as possible and without any mechanical and cost-intensive conversion of the ECC system, allows multifunctional use and above all without the need for a circulation stop and the risks and dangers to the patient involved with it, by making use of the options of other open and/or closed ECC systems that a minimized ECC system (particularly a minimized ECC system without automatic negative pressure and volume compensation) cannot provide.

Additionally, it shall provide the possibility to eliminate above all most of the regularly appearing air bubbles in the venous line, without the need for stopping the circulation every time for this purpose and without the risk to the patient increasing.

According to the invention, a special reservoir, particularly a venous cardiotomy reservoir, is proposed, which consists of a bottom form-stable part and a flexible part arranged on top of it. Due to its form stability, the bottom part has the ability to integrate or additionally accommodate one or several level sensors or means to hold the same, such as adhesive elements, without that these change their position or even get lost while the volumetric capacity of the reservoir changes. The part arranged on top of the other one is of flexible design. Due to its design (for instance as bellows) and/or due to its material quality and/or due to additional means, such as positive or non-positive elements, for instance spring suspensions, it seeks to expand and increase its volumetric capacity for the priming solution and/or for the collection of blood. The above-mentioned sensor arrangement for level monitoring is unaffected of this. Besides, preferably in the top region of the reservoir, one or several means are provided for optional opening the latter to the atmosphere. These means can be implemented, for instance, by sealing stoppers, clamping tubes or other venting elements.

With the invention, a multifunctionally usable reservoir was created that features a flexibly adjustable volumetric capacity being suitable for minimized, closed and open ECC systems. In this way, first of all the advantages of a minimized ECC system with the advantages particularly of minimal blood dilution and low blood/foreign surface contact can be used. Only by manual or automatic repositioning of clamps or by opening and closing of system components, thus without any additional efforts for a change of the construction (conversion) or for the exchange of the ECC system and without the risks and dangers to the patient known to be connected with it, such as air embolism, one and the same device with the proposed reservoir can be used universally if required with the option of other open and/or closed ECC systems, which the said minimized ECC system is unable to provide.

That way, there is no need for any mechanical efforts for the conversion or for the substitution of the ECC system, which are not only cost-intensive and require the provision of different, application-specific ECC systems or parts of the same, but which above all would have the consequence of a health-risking circulatory stop of the patient to be forced for it.

Such a universally usable ECC system can be used to advantage multifunctionally, for instance, in mobile emergency aid and in clinical use under the options of all known open, closed or minimized ECC systems.

It may be kept ready, for instance, for an emergency completely set up and filled with priming solution as mobile rescue perfusion system (minimized ECC system, often erroneously called ECMO).

If there is no emergency application taking place, the same ECC system can be used, for instance, for a scheduled bypass or cardiac valve surgery optionally as minimized ECC system with automatic negative pressure and volume compensation, as closed ECC system, or as open ECC system. If it is used in an emergency, in particular the emergency patient connected to the minimized ECC system can be transported to the operating room and the emergency operation can be carried out immediately without the use of another heart-lung machine being absolutely necessary.

The functionality of such a multifunctional ECC system has already been tested successfully on a specially made test setup in the animal-experimental research laboratory of the Medical Center of the Friedrich-Schiller University Jena.

Should macro bubbles appear in the venous line of the ECC system, it is advantageous if the blood is diverted from a bypass of the reservoir through the same by appropriate closing and opening of clamps, so that the air bubbles are eliminated there. Here, a sensor for bubble detection is useful, which—for the automatic diversion of blood through the reservoir—has connection to the corresponding clamps (electrical tube clamps) in the bypass line and the supply line of the reservoir.

If macro bubbles appear in the arterial line of the ECC system, it is advantageous if the blood is diverted through an arterial filter by appropriate opening and closing of clamps so that the air bubbles are eliminated therein. Here, a bubble sensor may be used, too, through which the corresponding clamps (electrical tube clamps) are operated automatically.

Also, it is useful if means are provided, for instance electrical tube clamps, to direct the blood flow automatically back through the reservoir.

Below, the invention shall be explained in more detail by way of design examples shown in the drawing.

The figures show:

FIG. 1: Schematic diagram of the actually known open ECC system

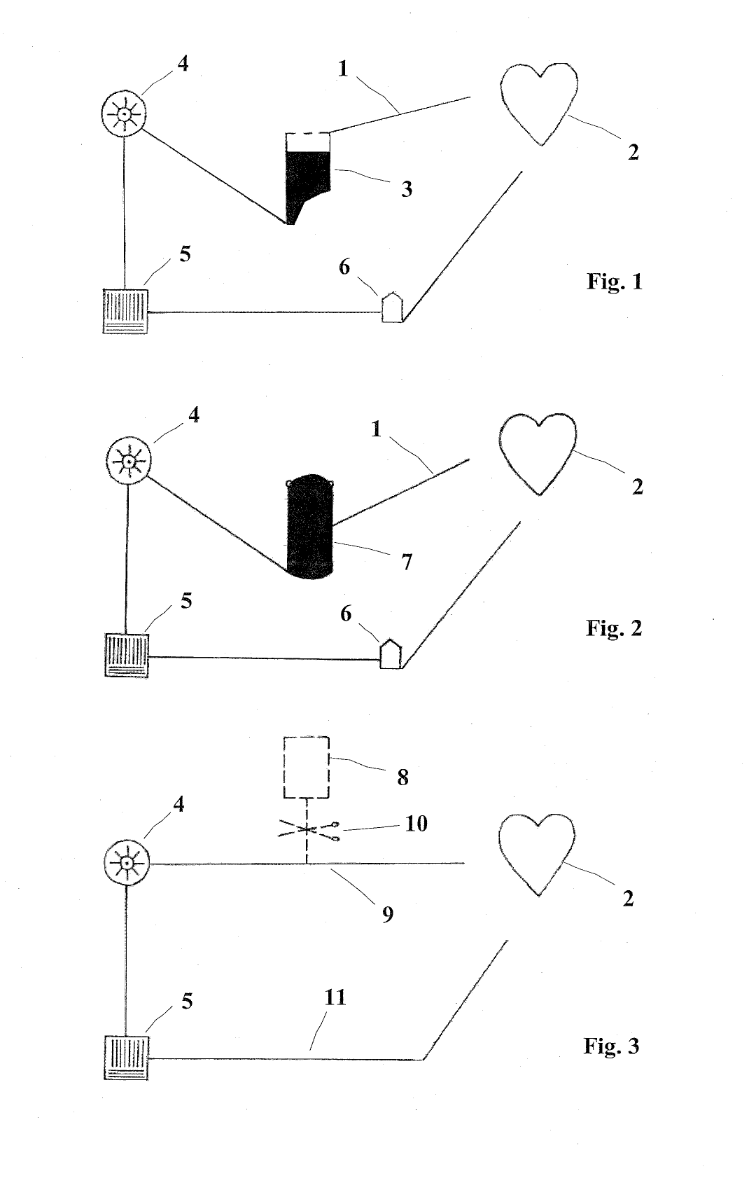

FIG. 2: Schematic diagram of the actually known closed ECC system

FIG. 3: Schematic diagram of the actually known minimized ECC system

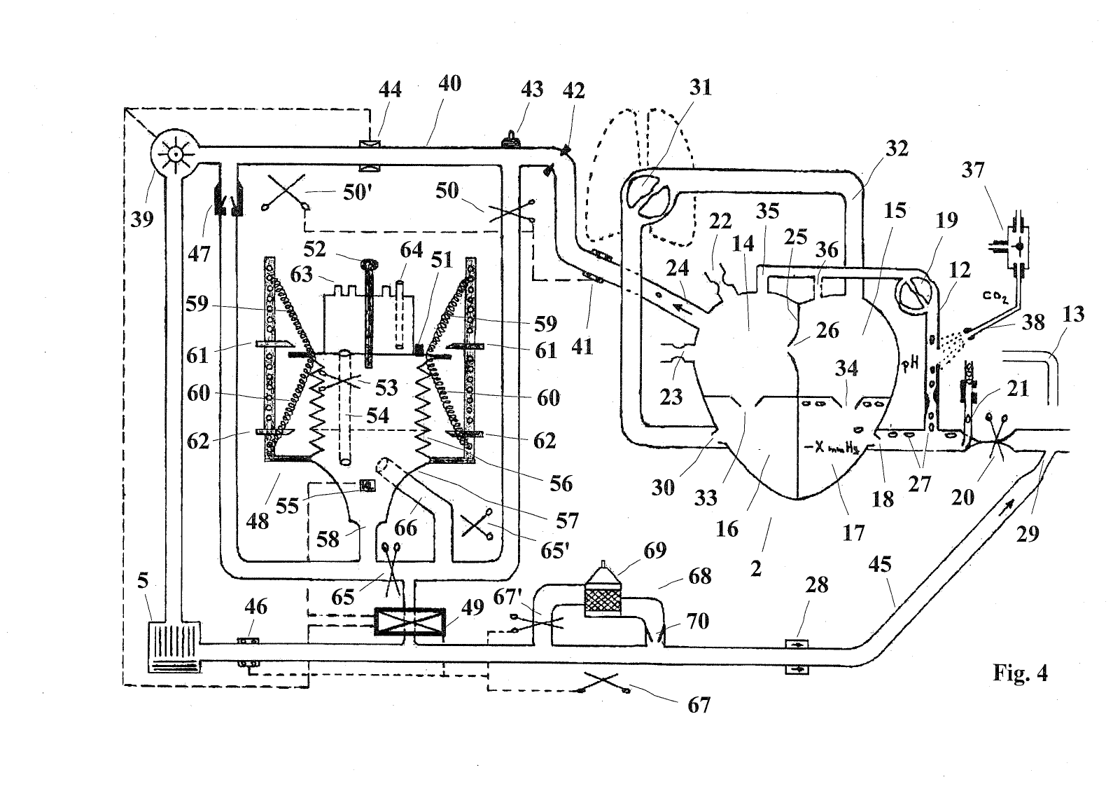

FIG. 4: Schematic diagram of a universally usable ECC system with the multifunctional reservoir suggested according to the invention

FIG. 1 shows the schematic setup of an actually known open ECC system. The blood coming from the patient is flowing passively through a venous tube line 1 from heart 2 into an open hard-shell reservoir 3. Here, the driving pressure is the central vein pressure and the hydrostatic pressure resulting from the difference in altitude between the heart 2 and the open hard-shell reservoir 3. Blood pump 4 aspirates the volume being present in the hard-shell reservoir 3 and pumps it into the artificial lung, the oxygenerator 5 with integrated heat exchanger. From there, the oxygen-rich blood gets back into the vascular system of the patient through an arterial filter 6.

FIG. 2 shows the schematic setup of an actually known closed ECC system. The blood coming from the patient flows passively from the heart 2 through the venous tube line 1 into a closed soft-bag reservoir 7. Here, the driving pressure is also the central vein pressure and the hydrostatic pressure resulting from the difference in altitude between the heart 2 and the closed soft-bag reservoir 7. The blood pump 4 aspirates the volume being in the soft-bag reservoir 7 and pumps it into the artificial lung, the oxygenerator 5 with integrated heat exchanger. From there, the oxygen-rich blood gets back into the vascular system of the patient through the arterial filter 6.

FIG. 3 shows the schematic setup of an actually known minimized ECC system. This ECC system is not only closed and thus without blood/air contact disadvantageous to the patient, but also the priming solution, the hemodilution (blood dilution) and the blood contact with foreign surfaces are minimized. This represents advantages to the patient, which are achieved, for instance, by a reservoir 8 (predominantly in the form of a bag, a bottle or a hard-shell reservoir) filled with blood or priming solution being arranged fully clamped out in the bypass and above a venous line 9 or not existing at all. When adding volume by manually opening a clamp 10, one must strictly take care that the reservoir 8 is not emptied and no air enters the system. The venous blood coming from the patient is actively aspirated directly by the blood pump 4 with short excessive negative pressure fluctuations possibly occurring, and pumped into the artificial lung, the oxygenerator 5 with integrated heat exchanger. From there, the oxygen-rich blood gets back directly into the vascular system of the patient through an arterial line 11.

FIG. 4 shows the schematic setup for a possible application of a universally usable ECC system with the suggested multifunctional reservoir using the example of an aortocoronary bypass surgery on the heart put out of action by cardioplegia. In this case example, the multifunctional ECC system (specifically termed ‘&ECC system”) is primarily used as minimized ECC system with automatic negative pressure and volume compensation.

A bypass 13 shall be sewed to a coronary artery 12 of the heart 2 put out of action by cardioplegia. The heart 2 is shown schematically in FIG. 4 with its right atrium 14, its left atrium 15, its right ventricle 16 and its left ventricle 17. The moderately stenosed coronary artery 12 leads from the aorta 18 (illustrated aortic valve with ascending aorta) to the capillary bed 19 of the heart 2. The aorta 18 is closed by an aortic clamp 20.

In the clamped aortic root, a cannula 21 is located, through which as so-called vent (ventricular venting cannula) a small volume of blood can be evacuated. For that, the negative pressure is limited to approx. −10 mmHg by a single-way valve, and the opening of this small cannula 21 is closed for the most part through the collapsing inner aortic wall. If vent evacuation is further increased, the single-way valve opens even more to the atmosphere in order to keep the negative pressure in the cannula 21 constant. Thus, that way, even more air is added to the evacuated blood so that this blood of the patient must be defoamed subsequently for re-use. The superior vena cava 22 and the inferior vena cava 23 through which venous blood gets into the right atrium 14 are partly collapsed due to heart dislocation or volume deficiency. Because of this, a centrifugal blood pump 39 does not get sufficient blood through a venous two-stage catheter 24 thus generating a high negative pressure difference. It is assumed that this strong negative pressure may be transmitted in weakened form up to the left heart and into the root of the aorta 18 (regarding this, in the Clinical Center of the Friedrich-Schiller University Jena incidentally further research work is planned on all described ECC systems, in particular a study on the said situation-related negative pressure values and on cardial air aspiration). This might occur by expansion of the atrial septum 25 and short opening of a functionally closed foramen ovale 26. That way, residual blood is evacuated for a short time from the left atrium 15 and the left ventricle 17 through the venous two-stage catheter 24 into the heart-lung machine, whereby, as illustrated in simplified form schematically in FIG. 4, air bubbles 27 (in this case, air bubbles with a high content of carbon dioxide) get into the left heart through the opened coronary artery. These get into the patient's circulation after the opening of aortic clamp 20 and might cause organ ischemias and cerebral infarcts. The risk of this air aspiration is minimized by the reservoir 48 used as negative pressure and volume compensation reservoir.

The blood oxygenated by the heart-lung machine is pumped again into the aorta 18 of the patient through a flow-through sensor 28 and an arterial cannula 29. For the sake of completeness and for a better understanding, FIG. 4 additionally shows in highly simplified form the patient's pulmonary valve 30 with the truncus pulmonalis, the capillary bed 31 of the lung, the pulmonary veins 32, the tricuspidal valve 33 in the right heart, the mitral valve 34 in the left heart as well as the sinus coronaries 35 and the vena oblique atrii sinistri 36. To improve the visibility for the operator and to minimize air embolisms, a volume of approx. 0.5 to 3.0 l/min carbon dioxide is blown onto the coronary anastomosis through a three-way cock 37 and a 1.2 mm or even better 1.5 mm wide button cannula 38, for which one should pay attention to sufficient humidity and greater variations of the myocardial pH value.

The speed-controlled centrifugal blood pump 39 actively evacuates blood predominantly from the right atrium 14 and the venae cavae 22, 23 through the two-stage catheter 24, the venous tube line 40, a venous bubble detector 41, a venous restrictor 42, an air bubble trap 43 and a pressure sensor 44 and pumps it into the oxygenator 5. Subsequently, the oxygen-enriched blood gets into the clamped aorta 18 through an arterial tube line 45, an arterial bubble detector 46, the flow sensor 28 and the arterial cannula 29. A flexible reservoir 48 is arranged in the vein bypass; it was filled with priming solution completely free of air by autologous priming at the beginning of the ECC, but nevertheless has not opened up fully and is closed to the atmosphere. The negative pressure in the venous tube line 40 measured by the pressure sensor 44 amounts to −40 mmHg, for instance, at full flow. If the negative pressure further increases e.g. by volume deficiency as a result of bleeding (and excretion through the kidneys) a single-way valve 47 opens at approx. −70 mmHg, and volume is aspirated from reservoir 48 for a short time. Furthermore, the centrifugal blood pump 39, which is controlled among others by the pressure sensor 44, receives the command to reduce its speed, which takes a certain time because of control algorithms and inertia, and/or an automatic clamp 49, which is also controlled by the pressure sensor 44, receives the command to open (this may also be done manually if need be), whereby part of the arterial blood flows back into the venous tube line 40 further thereby compensating the negative pressure or filling up the reservoir 48 again. When the volume deficiency is compensated and the negative pressure in the venous tube line 40 is within the desired range again, the speed of the centrifugal pump 39 is increased again or the automatic clamp 49 closed, which in the ideal case is done automatically. In reservoir 48, due to its endeavor to expand and in this way increase its volumetric capacity (instead of collapsing like a soft-bag reservoir), a negative pressure of −45 mmHg, for instance, exists so that a negative pressure difference of −25 mmHg for the hydrostatic pressure is to be added, which also acts on the single-way valve 47, so that it opens at −70 mmHg. Acting as negative pressure reservoir, the reservoir 48 is primarily filled with priming solution serving for the automatic negative pressure and volume compensation and being arranged in the bypass of the venous tube line 40. It is activated automatically only as described, when an excessive negative pressure arises; otherwise, it does not take part in the artificial blood circulation. If it is desirable to have a little and slow volume compensation only, the venous bypass tube line can be clamped out partly at single-way valve 47. If the negative pressure is in the normal range during the entire ECC time and no volume had to be substituted automatically, the priming solution of reservoir 48 is discarded. If the bubble detector 41 detects air bubbles in the venous tube line 40, and a certain regularity of air aspiration is to be expected, especially because of complications or an extension of the operation, the venous blood with the air bubbles can be directed through reservoir 48 by manually or automatically (controlled by bubble detector 41) repositioning a clamp 50 (indicated by a clamp 50′). In this way, the minimized ECC system with automatically switchable reservoir 48 for negative pressure and volume compensation turns within seconds into a closed ECC system with one and the same reservoir 48 as closed venous cardiotomy reservoir directly in venous tube line 40. The partly collapsed reservoir 48 then supports venous drainage without, however, generating excessive negative pressures. Now, most of the air bubbles rise in reservoir 48 gathering at the highest point. If a regular air aspiration into the venous tube line 40 cannot be stopped even after several minutes, there is a possibility to open reservoir 48 without any problems to the atmosphere, for instance, with a screw plug 51, a sealing plug 52, or a clamp 53 at a tube 54. That way, a closed ECC system with the reservoir 48 being used as closed flexible reservoir turns into an open ECC system with again one and the same reservoir 48, now used as open, form-stable reservoir, from which the air bubbles can escape now automatically and in which the fill level can be monitored through a level sensor 55.

In this case, the reservoir 48 consists of a vertical transparent bellows 56 in the middle section and a transparent form-stable part 57 in the bottom section, to which an adhesive part for holding the level sensor 55, for instance an ultrasonic level sensor, was attached and which is tapered at the bottom to form a reservoir outlet 58. The endeavor of the reservoir 48 to expand can be increased by springs 59 or impeded by springs 60 that way increasing or decreasing the negative pressure in the reservoir or the negative pressure difference required by the centrifugal pump 39 for the aspiration of blood and/or priming solution from the reservoir 49. As desired, the expansion of the reservoir 48 and thus the maximum fill volume may also be limited each by an occluder 61 or the contraction of an occluder 62. The limitation of the contraction may be useful, for instance, if the reservoir 48 is operated as closed reservoir and if it contains some air. Through ports 63, for instance, suction blood from the operating area or priming solution can be fed in. This volume can flow through filters and defoamers and, for instance, by opening the sealing plug 52 into the part of the reservoir located underneath, or above all, for the purpose of processing the suction blood, it can be evacuated again through an opening 64 (which at the same time may constitute a said opening to the atmosphere). By repositioning a clamp 65 to a reservoir inlet 66 (indicated by a clamp 65′) the arterial blood and/or the priming solution can flow directly from the reservoir 48 into the venous tube line 40, when the automatic clamp 49 is opened. In some situations, it is also useful to put the clamps onto other places of the tube system depending on the user's experience. In the case of the detection of micro or macro bubbles by the bubble detector 46, for instance, the blood and/or the priming solution could be directed through an arterial filter path 68 with an arterial filter 69 and a single-way valve 70 for debubbling. Also, the blood and/or the priming solution could flow back into the reservoir 48 by opening the automatic clamp 49 and correspondingly open/close the clamps 65 and 65′.

### LIST OF USED REFERENCE CHARACTERS

1, 9, 40—Venous tube line
2—Heart
3—Hard-shell reservoir
4—Blood pump
5—Oxygenator
6, 69—Arterial filter
7—Soft-bag reservoir
8, 48—Reservoir
10, 53, 50, 50′, 65, 65′, 67, 67′—Clamps
11, 45—Arterial tube line
12—Coronary artery
13—Bypass
14, 15—Right or left atrium
16, 17—Right or left ventricle
18—Aorta
19—Capillary bed
20—Aortic clamp
21—Cannula
22, 23—Vena carva
24—Two-stage catheter
25—Atrial septum
26—Foramen ovale
27—Air bubbles
28—Flow-through sensor
29—Arterial cannula
30—Pulmonary valve
31—Capillary bed of the lung
32—Pulmonary veins
33—Tricuspidal valve
34—Mitral valve
35—Sinus coronarius
36—Vena oblique atrii sinistri
37—Three-way cock
38—Button cannula
39—Centrifugal blood pump
41—Venous bubble detector
42—Venous restrictor
43—Air bubble trap
44—Pressure sensor
46—Arterial air bubble detector
47, 70—Single-way valves
49—Automatic clamp
51—Screw plug
52—Sealing plug
54—Tube
55—Level sensor
56—Bellows
57—Form-stable part
58—Reservoir outlet
59, 60—Springs
61, 62—Occluders
63—Ports
64—Opening
66—Reservoir inlet
68—Filter path

1. Extracorporeal circulation system (ECC system), particularly for a multifunctional heart-lung bypass and for the minimization of air embolisms, preferably equipped with a centrifugal blood pump, an oxygenator and a reservoir, in particular a venous cardiotomy reservoir, for blood and/or priming solution, characterized in that, above all for the purpose of an optional application as minimized ECC system with completely manually clamped out reservoir in the bypass of the venous line, as minimized ECC system with automatically activatable reservoir in the bypass of the venous line, as closed ECC system with closed reservoir directly in the venous line, or as open ECC system with a reservoir open to the atmosphere, a reservoir (48) is provided, which in its bottom section consists of a form-stable part (57) that can at least hold one level sensor (55) and that is tapered preferably to the reservoir outlet (57), and which, at least in its section located above the form-stable part (59), has a flexible part (56) that due to its design and/or its material properties and/or additional means (5) shows the endeavor to expand and increase its volumetric capacity for the holding of priming solution and/or blood and that in its top section has at least one element (51, 52, 53 and 54, 64) or place for optional opening to the atmosphere.

2. Extracorporeal circulation system according to claim 1, characterized in that the form-stable part (57) is conically tapered to the reservoir outlet (58).

3. Extracorporeal circulation system according to claim 1, characterized in that one or several level sensors (55) for acquiring or monitoring the fill level of blood and/or priming solution are provided on or in the form-stable part (57).

4. Extracorporeal circulation system according to claim 1, characterized in that the flexible part (56) is designed as vertical bellows.

5. Extracorporeal circulation system according to claim 1, characterized in that springs (59, 60) fixed to the flexible part (56) are provided, which impede, initiate or support the endeavor of the flexible part (56) to expand and increase its volumetric capacity for the holding of priming solution and/or blood.

6. Extracorporeal circulation system according to claim 1, characterized in that stop elements (61, 62) are provided, which locally limit the expanding movement of the flexible part (56).

7. Extracorporeal circulation system according to claim 1, characterized in that the at least one element for the optional opening to the atmosphere consists of a sealing plug (52).

8. Extracorporeal circulation system according to claim 1, characterized in that the at least one element for the optional opening to the atmosphere consists of a screw plug (51).

9. Extracorporeal circulation system according to claim 1, characterized in that the at least one element for the optional opening to the atmosphere consists of a tube (54) with a detachable clamp (53).

10. Extracorporeal circulation system according to claim 1, characterized in that the at least one element for the optional opening to the atmosphere is constructed at the same time as fill-in element (64) for blood, priming solution and other fluid media.

11. Extracorporeal circulation system according to claim 1, characterized in that for the purpose of detecting and eliminating air bubbles a bubble detector (41) is provided in the venous line (40) of the ECC system, which is in connection with corresponding clamps, preferably electrical clamps (50, 50′), in a supply line and in a bypass line of the reservoir (48), in order to eliminate the air bubbles to the greatest possible extent through directing them through the reservoir (48) by opening or closing the clamps (50, 50′) accordingly.

12. Extracorporeal circulation system according to claim 1, characterized in that for the purpose of detecting and eliminating air bubbles a bubble detector (46) is provided in the arterial line (45) of the ECC system, which is in connection with corresponding clamps, preferably electrical clamps (67, 67′), in the arterial line (45) as well as in a bypass line (68) containing an arterial filter (69), in order to eliminate the air bubbles by directing them through the arterial filter (69) by opening or closing the clamps (67, 67′) accordingly.

13. Extracorporeal circulation system according to claim 1, characterized in that means are provided, for instance at least one electrical tube clamp (49), in order to direct the blood flow automatically back through the reservoir (48).
